(19) United States
(12) Patent Application Publication
(10) Pub. No.: US 2007/0129450 A1
(43) Pub. Date: Jun. 7, 2007

(54) Process for producing variable syngas compositions

(76) Inventors: Scott Donald Barnicki, Kingsport, TN (US); Nathan West Moock, Kingsport, TN (US); William Lewis Trapp, Kingsport, TN (US)

Correspondence Address: ERIC D. MIDDLEMAS;EASTMAN CHEMICAL COMPANY, P. O. BOX 511, KINGSPORT, TN, 37662-5075, US

(21) Appl. No.: 11/282,261

(22) Filed: Nov. 18, 2005

(51) Int. Cl. F02C 9/00; C07C 27/06; C01B 3/32

(52) U.S. Cl. 518/704; 60/781; 60/39.12; 60/39.13; 518/702; 518/703; 518/705; 48/198.7; 48/198.3

(57) ABSTRACT

Disclosed is a process for the production of a variable syngas composition by gasification. Two or more raw syngas streams are produced in a gasification zone having at least 2 gasifiers and a portion the raw syngas is passed to a common water gas shift reaction zone to produce at least one shifted syngas stream having an enriched hydrogen content and at least one unshifted syngas stream. The shifted and the unshifted syngas streams are mixed downstream of the water gas shift zone in varying proportions produce blended and unblended synthesis gas streams in a volume and/or composition that may vary over time in response to at least one downstream syngas requirement. The process is useful for supplying syngas from multiple gasifiers for the variable coproduction of electrical power and chemicals across periods of peak and off-peak power demand.

### FIELD OF THE INVENTION

This invention relates to a process for the production of two or more synthesis gas streams of variable compositions and volumes. More particularly, this invention relates to a process wherein at least a portion of two or more synthesis gas streams from a gasification zone is passed to a water gas shift zone to enhance its hydrogen content, and the shifted and unshifted streams are mixed downstream of the water gas shift zone to produce at least one blended syngas stream having a volume and/or composition which can vary over time.

### BACKGROUND OF THE INVENTION

The high price and diminishing supply of natural gas and petroleum has caused the chemical and power industry to seek alternative feedstocks for the production of chemicals and the generation of electrical power. Coal and other solid carbonaceous fuels such as, for example, petroleum coke, biomass, paper pulping wastes, by contrast, are in great abundance and relatively inexpensive, and are logical materials for the art to investigate as alternative feedstock sources. Coal and other solid carbonaceous materials can be gasified, i.e., partially combusted with oxygen, to produce synthesis gas (also referred to hereinafter as “syngas”), which can be cleaned and used to produce a variety of chemicals or burned to generate power. Gasification processes typically produce a synthesis gas with a molar ratio of H₂ to CO of about 0.4/1 to 1.2/1, together with lesser volumes of CO₂, H₂S, methane and other inerts.

Different applications, however, require different H₂/CO ratios to utilize the syngas raw material efficiently. For example, Fischer-Tropsch and methanol reaction stoichiometries require a 2/1 molar ratio of H₂/CO, synthetic natural gas production requires 3/1, acetic acid synthesis requires 1/1, syngas for ammonia or hydrogen production require hydrogen only. This ratio can be adjusted by means known in the art, e.g., via the water gas shift reaction wherein carbon monoxide is reacted with water to produce hydrogen and carbon dioxide. This approach is not satisfactory, however, when there are multiple, different, downstream requirements for syngas. For example, when designing an integrated process to produce syngas with varying H₂/CO ratio requirements such as found in chemical and power coproduction facility, one approach is to shift all syngas from a gasification zone to the highest required H₂/CO ratio, i.e. overshifting some fraction of the gas. The overshifting approach, however, imparts an energy penalty to those processes not requiring syngas with a high hydrogen to carbon molar ratio. Because the water gas shift reaction is exothermic, a portion of the chemical energy in the syngas (equivalent to the enthalpy of reaction of the water-gas shift reaction) is converted to thermal energy during the shift reaction. Power production, therefore, is maximized by utilizing unshifted gas. For example, shifting to a 2/1H₂/CO molar ratio can result in a loss of about 3-12% of the chemical energy compared to the unshifted gas. The extent of the loss is dependent on the initial H₂/CO molar ratio of the syngas. Hence, mole for mole, shifted gas has a lower energy content than unshifted gas.

An integrated gasification combined cycle (abbreviated herein as “IGCC”) power plant typically consists of a fuel (usually coal or pet coke) gasification block and a combined cycle power block. The combined cycle and power block are essentially identically to that used with natural gas fuels. The generation and utilization of syngas from a gasification process, however, is much more complicated than drawing fuel from a natural gas pipeline. The solids grinding and preparation, gasification, ash handling, gas cooling, and sulfur removal steps associated with an IGCC are capital intensive, and difficult and costly to shut down and start up frequently. IGCC power plants are designed to operate continuously with limited turndown capacity and inherently favor substantially continuous base-load operation. Even if the gasification block could be turned off as readily as pipeline-based natural gas, idling of the gasifier block and would result in under utilization of the assets and a prohibitive economic penalty on power production. Thus, there is a mismatch between the variable power production ability of the combined cycle block and the required base-loaded operation of the gasification block. IGCC units are considered in the art as base-load units, meaning that they lack the ability to dispatch to intermediate load factors. In many power markets, the price of power can vary by a factor of 2 or more between peak power demand periods and periods of low power demand such as, for example between night and day. Reliance on base load operation may severely limit the economic viability of power production via IGCC. In fact, the most economic solution may be to produce no power during off-peak periods. Thus, there is a need for a IGCC process that can produce higher value products than electricity with available syngas during off-peak power times.

The potential benefits of the coproduction of chemicals with power have been well-studied and are discussed, for example, in “Clean Coal Technology: Coproduction of Power, Fuels, and Chemicals”, Topical Report 21, September, 2001, U.S. Department of Energy, and Gray and Tomlinson, “Coproduction: A Green Coal Technology”, Mitretek Technical Report MP 2001-28, March, 2001. Numerous variations have been proposed in the prior art to address the issue of chemical and power coproduction. A common approach is to operate the gasification block at an essentially constant base-load capacity factor. The crude syngas thus generated is cleaned to remove the majority of the sulfurous compounds and other impurities, followed by feeding the cleaned syngas to a so-called partial-conversion, “once-through” (no gas recycle) chemical synthesis reaction, with the unconverted syngas burned for direct base load power generation. The synthesized chemical is stored and later used as fuel for gas turbine-steam turbine combined cycle system during the peak demand periods or sold when in excess. Co-produced chemicals exemplified in the art are ammonia, methanol, dimethyl ether, and Fischer-Tropsch hydrocarbons.

Examples of such partial conversion processes with chemical coproduction are described, for example, in U.S. Pat. No. 4,566,267 for ammonia coproduction, U.S. Pat. No. 5,392,594 for methanol, U.S. Pat. Nos. 3,986,349 and 4,092,825 for Fischer-Tropsch hydrocarbons, and U.S. Pat. No. 4,341,069 for dimethyl ether coproduction. Additional discussion of chemical and power coproduction may be found, for example, in Weber et al “Methanol Coproduction: Strategies for Effective Use of IGCC Power Plants”, Proceedings of the American Power Conference (1988), 50, pp. 288-93. “Once through” chemical processes, however, enable production of relatively small amounts of chemicals. For example, a “once-through” methanol process typically utilizes about 12-30% of the carbon monoxide/hydrogen feed gas and, thus, do not efficiently use the available syngas feedstock. Because a limited amount of chemical product can be co-produced, a significant base-load power operation is still required. Moreover, such “once through” processes lack of economy of scale for chemical production and often result in a high capital cost.

The utilization of unshifted syngas for chemical synthesis often severely limits the maximum chemical production that can be achieved. For example, the synthesis of methanol, dimethyl ether, and Fischer-Tropsch hydrocarbons consumes two moles of H₂ per mole of CO, and it is readily apparent that, even if H₂ conversion is complete, this stoichiometric requirement will limit the conversion of an unshifted syngas stream. Since only a limited fraction, typically about 50%, of the available hydrogen is converted in the once-through synthesis mode, the process will convert a maximum of only about 25% of the available syngas to a chemical product. Chemical equilibrium and kinetics limitations further constrain the potential achievable conversions at compositions, temperatures, and pressures at which the reactions may be carried out in practice.

In addition to the deficiencies described above, the methods and processes in the art above do not adequately address the problem of producing multiple syngas compositions for downstream syngas requirements such as, for example, chemical and power coproduction, in which the volume and/or composition of the syngas required for each function may vary over time. Schemes relying on continuous once-through chemical and power coproduction require substantial base-load operation at all times because of stoichiometric limitations of the chemical reaction and can result in high capital requirements. For chemical and power coproduction, a method of variable power production is needed that optimizes the amount of syngas that is shifted during periods of coproduction such that the energy penalty to power production is minimized, capital costs are reduced, and the highest thermal efficiency of power cycle is maintained during power production, while converting unused syngas fuels to chemicals at the highest stoichiometric and capital efficiency during chemical production. Finally, a method is needed to minimize shift reactor volume required for coproduction scenarios with multi-gasifier configurations.

### SUMMARY OF THE INVENTION

We have discovered that multiple syngas streams having a time variant composition and volume can be efficiently produced by using two or more gasifiers to supply raw syngas to a central water gas shift zone, shifting a portion of the raw syngas, and blending the shifted and unshifted gas steams downstream of the water gas shift zone in proper proportions to meet one or more downstream syngas requirements. Accordingly, the present invention provides a process for producing variable syngas compositions, comprising:
(a) reacting an oxidant stream with a carbonaceous material in a gasification zone comprising at least 2 gasifiers to produce at least 2 raw syngas streams comprising carbon monoxide, hydrogen, carbon dioxide, and sulfur-containing compounds,
(b) passing a portion of at least one of the raw syngas streams from step (a) to a common water-gas shift reaction zone to produce at least one shifted syngas stream (i) having an enriched hydrogen content, and at least one unshifted syngas stream (ii), comprising a remaining portion of the raw syngas streams; and
(c) blending the shifted syngas stream (i) with a portion of the unshifted syngas stream (ii) to produce at least one blended syngas stream (iii) and at least one unblended syngas stream (iv) comprising a remaining portion of unshifted syngas stream (ii) wherein the blended syngas stream is produced in a volume and/or composition that varies in response to at least one downstream syngas requirement.
   The instant invention provides for at least 2 gasifiers connected to a common or shared water gas shift reaction zone in which a portion of the raw syngas from these gasifiers may be directed to produce at least one shifted syngas stream having an enriched hydrogen content and at least one unshifted gas stream comprising the remaining portion of the raw syngas stream. Another aspect of the instant invention is the blending of the shifted syngas stream with all or a portion of the unshifted syngas stream downstream of the gasification zone and water gas shift reaction zone to produce blended and unblended syngas streams. Redundant gas cooling and acid gas removal zones are provided for shifted and unshifted syngas streams of variable composition, consistent with maximum scalable train size, such that the zones can be fed via a syngas header system downstream of the gasification zone and water gas shift reaction zone. The composition of these syngas streams may be varied over time according to at least one downstream syngas requirement such as, for example, a feedstock need of a least one chemical process, a fuel need of at least one power plant, or a combination thereof.

In one embodiment of the invention, for example, the blended syngas stream may be passed to a methanol or dimethyl ether producing zone and the unblended syngas stream passed to a power producing zone to produce electrical power. Steam may be produced from the water gas shift reaction zone by the recovery of heat from the shifted syngas stream and a portion of that steam may be combined with the raw syngas to provide a wet syngas for the water gas shift reaction. Thus, the present invention also provides a process for producing variable syngas compositions, comprising:
(a) reacting an oxidant stream with coal or petroleum coke in a gasification zone comprising at least 2 gasifiers to produce at least 2 raw syngas streams comprising carbon monoxide, hydrogen, carbon dioxide, and sulfur-containing compounds,
(b) passing a portion of at least one of the raw syngas streams from step (a) to a common water-gas shift reaction zone to produce at least one shifted syngas stream (i) having a molar ratio of hydrogen to carbon monoxide of about 1:1 to about 20:1, and at least one unshifted syngas stream (ii), comprising a remaining portion of the raw syngas streams;
(c) generating steam in the water-gas shift reaction zone by recovery of heat from the shifted syngas stream (i);
(d) combining a portion of the steam from step (c) with the portion of one or more raw syngas streams before passing to the water-gas shift reaction zone;
(e) blending the shifted syngas stream (i) with a portion of the unshifted syngas stream (ii) to produce at least one blended syngas stream (iii) and at least one unblended syngas stream (iv) comprising a remaining portion of unshifted syngas stream (ii); and
(f) passing blended gas stream (iii) to a methanol or dimethyl ether producing zone and unblended gas stream (iv) to a power producing zone.

The blended and unblended syngas stream may be passed to a methanol producing zone and a power producing zone and can be produced in volumes that vary in response to peak and off-peak power demands. Thus, another embodiment of our invention is a process for producing variable volumes of power and methanol, comprising:
(a) reacting an oxidant stream with coal or petroleum coke in a gasification zone comprising at least 2 gasifiers to produce at least 2 raw syngas streams comprising carbon monoxide, hydrogen, carbon dioxide, and sulfur-containing compounds,
(b) passing a portion of at least one of the raw syngas streams from step (a) to a common water-gas shift reaction zone to produce at least one shifted syngas stream (i) having an enriched hydrogen content, and at least one unshifted syngas stream (ii), comprising a remaining portion of the raw syngas streams;
(c) blending the shifted syngas stream (i) with up to 100 volume percent of the unshifted syngas stream (ii) to produce at least one blended syngas stream (iii) and a remaining portion of the unshifted syngas stream (ii);
(d) producing methanol by passing the blended gas stream (iii) from step (c) to a methanol producing zone; and
(e) passing the remaining portion of unshifted syngas stream (ii) to a power producing zone to produce electrical power;
wherein the blended syngas stream is produced in a volume that varies in response to periods of peak and off-peak power demands on the power producing zone.
   The syngas is consumed in a methanol producing zone and a power producing zone in which the syngas requirement varies cyclically and substantially out of phase. In one embodiment, for example, during a period of off-peak power demand, up to 100 percent of the unshifted syngas is blended with the shifted syngas to produce at least one blended syngas stream that is used to produce methanol. Alternatively, during periods of peak power demand, less of the raw syngas is shifted and can be directed to a power plant to produce electrical power.

### BRIEF DESCRIPTION OF DRAWINGS

FIG. 1 illustrates a schematic flow diagram for one embodiment for producing syngas of variable composition and volumes.

### DETAILED DESCRIPTION

The present invention provides for at least 2 gasifiers connected to a common or shared water gas shift reaction zone in which a portion of the raw syngas from these gasifiers may be directed to produce at least one shifted syngas stream having an enriched hydrogen content and at least one unshifted gas stream comprising the remaining portion of the raw syngas streams. The shifted and remaining portion of the unshifted syngas can be blended downstream of the water-gas shift reaction zone to produce blended and unblended syngas streams. Thus, in a general embodiment, the present invention provides a process for producing variable syngas compositions, comprising:
(a) reacting an oxidant stream with a carbonaceous material in a gasification zone comprising at least 2 gasifiers to produce at least 2 raw syngas streams comprising carbon monoxide, hydrogen, carbon dioxide, and sulfur-containing compounds,
(b) passing a portion of at least one of said raw syngas streams from step (a) to a common water-gas shift reaction zone to produce at least one shifted syngas stream (i) having an enriched hydrogen content, and at least one unshifted syngas stream (ii), comprising a remaining portion of said raw syngas streams; and
(c) blending said shifted syngas stream (i) with a portion of said unshifted syngas stream (ii) to produce at least one blended syngas stream (iii) and at least one unblended syngas stream (iv) comprising a remaining portion of unshifted syngas stream (ii),
wherein said blended syngas stream in produced in a volume and/or composition that varies in response to at least one downstream syngas requirement.
   The volume and composition of the blended and unblended syngas streams can be varied over time to satisfy one or more downstream syngas requirements such as, for example, a feedstock requirement for a methanol plant, a fuel for a power plant, or a combination thereof. According to our invention, carbonaceous materials can be continuously reacted with oxygen in one or more gasifiers to produce syngas at a substantially constant rate. The term “substantially constant rate”, as used herein, is understood to mean that the gas is provided continuously in an uninterrupted manner and at a constant level. “Substantially constant rate”, however, is not intended to exclude normal interruptions that may occur because of, for example, maintenance, start-up, or scheduled shut-down periods. For the purposes of this invention, the term “sulfur” and “sulfur-containing compound” are synonymous and refer to any sulfur-containing compound, either organic or inorganic in nature. Examples of such sulfur-containing compounds are exemplified by hydrogen sulfide, sulfur dioxide, sulfur trioxide, sulfuric acid, elemental sulfur, carbonyl sulfide, mercaptans, and the like. Although the syngas, comprising carbon dioxide, carbon monoxide, and hydrogen, of the instant invention may be provided by any of a number of methods known in the art such as, for example, steam or carbon dioxide reforming of carbonaceous materials such as natural gas or petroleum derivatives, it is preferably obtained by partial oxidation or gasification of carbonaceous materials, such as petroleum residuum, bituminous, subbituminous, and anthracitic coals and cokes, lignite, oil shale, oil sands, peat, biomass, petroleum refining residues or cokes, and the like.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, each numerical parameter should at least be construed in light of the number of reported significant digits and by applying ordinary rounding techniques. Further, the ranges stated in this disclosure and the claims are intended to include the entire range specifically and not just the endpoint(s). For example, a range stated to be 0 to 10 is intended to disclose all whole numbers between 0 and 10 such as, for example 1, 2, 3, 4, etc., all fractional numbers between 0 and 10, for example 1.5, 2.3, 4.57, 6.113, etc., and the endpoints 0 and 10. Also, a range associated with chemical substituent groups such as, for example, “C₁ to C₅ hydrocarbons”, is intended to specifically include and disclose C₁ and C₅ hydrocarbons as well as C₂, C₃, and C₄ hydrocarbons.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

As used in the specification and the appended claims, the singular forms “a,” “an” and “the” include their plural referents unless the context clearly dictates otherwise. For example, references to a “syngas stream,” or a “gasifier,” is intended to include the one or more syngas streams, or gasifiers. References to a composition or process containing or including “an” ingredient or “a” step is intended to include other ingredients or other steps, respectively, in addition to the one named.

By “comprising” or “containing” or “including”, we mean that at least the named compound, element, particle, or method step, etc., is present in the composition or article or method, but does not exclude the presence of other compounds, catalysts, materials, particles, method steps, etc, even if the other such compounds, material, particles, method steps, etc., have the same function as what is named, unless expressly excluded in the claims.

It is also to be understood that the mention of one or more method steps does not preclude the presence of additional method steps before or after the combined recited steps or intervening method steps between those steps expressly identified. Moreover, the lettering of process steps or ingredients is a convenient means for identifying discrete activities or ingredients and the recited lettering can be arranged in any sequence, unless otherwise indicated.

The process of the invention includes reacting an oxidant stream with a carbonaceous material in a gasification zone comprising at least 2 gasifiers to produce at least 2 raw syngas streams comprising carbon monoxide, hydrogen, carbon dioxide, and sulfur-containing compounds. Typically, the 2 or more gasifiers are sized to supply at least 90% of the maximum capacity fuel requirements of a power-producing zone. Any one of several known gasification processes can be incorporated into the method of the instant invention. These gasification processes generally fall into broad categories as laid out in Chapter 5 of “Gasification”, (C. Higman and M. van der Burgt, Elsevier, 2003). Examples are moving bed gasifiers such as the Lurgi dry ash process, the British Gas/Lurgi slagging gasifier, the Ruhr 100 gasifier; fluid-bed gasifiers such as the Winkler and high temperature Winkler processes, the Kellogg Brown and Root (KBR) transport gasifier, the Lurgi circulating fluid bed gasifier, the U-Gas agglomerating fluid bed process, and the Kellogg Rust Westinghouse agglomerating fluid bed process; and entrained-flow gasifiers such as the Texaco, Shell, Prenflo, Noell, E-Gas (or Destec), CCP, Eagle, and Koppers-Totzek processes. The gasifiers contemplated for use in the process may be operated over a range of pressures and temperatures between about 1 to about 103 bar absolute (abbreviated herein as “bara”) and 400° C. to 2000° C., with preferred values within the range of about 21 to about 83 bara and temperatures between 500° C. to 1500° C. Depending on the carbonaceous or hydrocarbonaceous feedstock used therein and type of gasifier utilized to generate the gaseous carbon monoxide, carbon dioxide, and hydrogen, preparation of the feedstock may comprise grinding, and one or more unit operations of drying, slurrying the ground feedstock in a suitable fluid (e.g., water, organic liquids, supercritical or liquid carbon dioxide). Typical carbonaceous materials which can be oxidized to produce syngas include, but are not limited to, petroleum residuum, bituminous, subbituminous, and anthracitic coals and cokes, lignite, oil shale, oil sands, peat, biomass, petroleum refining residues, petroleum cokes, and the like.

The oxidant stream may comprise pure molecular oxygen or another suitable gaseous stream containing substantial amounts of molecular oxygen and is charged to the gasifier, along with the carbonaceous or hydrocarbonaceous feedstock. The oxidant stream may be prepared by any method known in the art, such as cryogenic distillation of air, pressure swing adsorption, membrane separation, or any combination therein. The purity of oxidant stream typically is at least 85 volume % oxygen; for example, the oxidant stream may comprise at least 95 volume % oxygen or, in another example at least 98 volume % oxygen.

The oxidant stream and the prepared carbonaceous or hydrocarbonaceous feedstock are introduced into at least 2 gasifiers wherein the oxidant is consumed and the feedstock is substantially converted into at least 2 synthesis gas (syngas) streams comprising carbon monoxide, hydrogen, carbon dioxide, water, and various impurities such as, for example, sulfur-containing compounds. Examples of impurities that the raw syngas streams may contain include hydrogen sulfide, carbonyl sulfide, methane, ammonia, hydrogen cyanide, hydrogen chloride, mercury, arsenic, and other metals, depending on the feedstock source and gasifier type. In addition to at least 2 gasifiers, the gasification zone may comprise high temperature gas cooling equipment, ash/slag handling equipment, gas filters, and scrubbers. The precise manner in which the oxidant and feedstock are introduced into the gasifier is within the skill of the art; typically, however, the gasifiers will be run continuously and at a substantially constant rate.

The water-gas shift reaction can be employed to alter the hydrogen to carbon monoxide molar ratio of the syngas and to provide the correct stoichiometry of hydrogen and carbon monoxide for chemical production. The process of the invention thus comprises passing at least one of the raw syngas streams from the gasification zone to a common water-gas shift reaction zone to produce at least one shifted syngas stream (i) having an enriched hydrogen content, and at least one unshifted syngas stream (ii) which comprises the remaining portion of the raw syngas streams. The term “a portion”, as used herein with respect to the raw syngas, is understood to mean a part or a fraction of a single raw syngas stream, a part of 2 or more raw syngas streams, or a part of the total raw syngas output from the gasification zone such as, for example, after mixing or combining multiple raw syngas streams in a central gas header or manifold. For example, about 1 to 100 volume % of one or more of the raw syngas streams, based on the total volume of the syngas streams, may be directed to the common water gas shift reaction zone. In another example, multiple raw syngas streams from the gasification zone can be combined in a central gas header or manifold and from about 1 to about 90 volume % of the combined stream, based on the total volume of the combined stream, may be passed to the common water gas shift reaction zone. The term “common”, as used herein, is intended to mean that the water gas shift reaction zone is connected to and shared by at least 2 gasifiers in contrast to each gasifier having a separate water gas shift zone for processing its syngas output, although more than one water gas shift reaction zone may be present. In one aspect of the invention, therefore, our process may include multiple water gas shift reaction zones as long as at least one of the water gas shift reaction zones is connected to and shared by at least 2 gasifiers.

A portion of at least one of the raw syngas streams is directed a common water-gas shift reaction zone in which the syngas undergoes the equilibrium-limited water-gas shift reaction in which carbon monoxide is reacted with water to produce hydrogen and carbon dioxide:
CO+H₂O←→CO₂+H₂
Typically the water-gas shift reaction is accomplished in a catalyzed fashion by methods known in the art. Advantageously the water gas shift catalyst is sulfur-tolerant. For example, such sulfur tolerant catalysts can include, but are not limited to, cobalt-molybdenum catalysts. Operating temperatures are typically 250° C. to 500° C.

The water-gas shift reaction may be accomplished in any reactor format known in the art for controlling the heat release of exothermic reactions. Examples of suitable reactor formats are single stage adiabatic fixed bed reactors; multiple-stage adiabatic fixed bed reactors with interstage cooling, steam generation, or cold-shotting; tubular fixed bed reactors with steam generation or cooling; and fluidized beds. Typically about 80-90% of the carbon monoxide will be converted to carbon dioxide and hydrogen in a single stage adiabatic reactor because of equilibrium limitations. If greater conversion is required (i.e., for hydrogen production), then additional stages with lower outlet gas temperatures may be used.

Because of the highly exothermic nature of the water-gas shift reaction, steam may be generated in the water-gas shift reaction zone by recovering heat from the shifted syngas stream (i) as it exits the water gas-shift reaction zone and before it is blended with the unshifted syngas stream (ii). The steam generated in the water-gas shift reaction zone may be directed to a common steam header and used as general utility steam. The raw syngas produced by gasification, however, often does not contain sufficient water in order to carry out the water gas shift reaction to the desired conversion. Alternatively, the steam generated in the water-gas shift reaction zone can be used to add sufficient water to the raw syngas entering the water-gas shift reaction zone by combining a portion of the steam with a the portion of one or more raw syngas streams from the gasification zone to produce at least one wet syngas stream and passing that wet syngas stream to the water-gas shift reaction zone. Typically, the molar ratio of water to carbon monoxide in the wet syngas stream is about 1.5:1 to about 3:1. Additional examples of water:carbon monoxide molar ratios that may be produced are 2:1 and 2.5:1.

The shifted syngas stream (i) can be blended with a portion of the unshifted syngas stream (ii) to produce at least one blended syngas stream (iii) and at least one unblended syngas stream (iv) which comprises the remaining portion of unshifted syngas stream (ii). In accordance with our invention, the volumes and composition of the blended and unblended syngas streams (iii) and (iv) respectively can be easily and quickly adjusted by changing one or more parameters including the portion of the raw syngas directed to the water-shift reaction zone, the conversion of CO in the water-gas shift reaction zone, and the portion of unshifted syngas stream (ii) blended with the shifted syngas stream (i). Blending of the shifted and unshifted gas streams may be accomplished by any means known to persons of ordinary skill in the art such as, for example, by passing the combined gas streams through a static mixer. The volume of the blended and unblended syngas streams (iii) and (iv) and/or the composition of the blended syngas stream (iii) may be varied over time in response to at least one downstream requirement such as, for example, a feedstock need of a least one chemical process, a fuel need of at least one power plant, or a combination thereof. For example, the blended (iii) and unblended (iv) syngas gas streams may be produced in volumes that vary periodically in response to at least one downstream syngas requirement. The term “periodically”, as used herein, is understood to have its commonly accepted meaning of “associated with or occurring in time intervals or periods”. The periods or time intervals may occur regularly, for example once every 24 hours, or irregularly.

In one embodiment, for example, the process of the invention further comprises passing the blended syngas stream (iii) to a chemical producing zone and the unblended syngas stream (iv) to a power producing zone which may be operated simultaneously or cyclically and substantially out of phase. The power producing zone comprises a means for converting chemical and kinetic energies in the syngas feed to electrical or mechanical energy, typically in the form of at least one turboexpander, also referred to hereinafter as “combustion turbine”. Typically, the power-producing zone will comprise a combined cycle system as the most efficient method for converting the energy in the syngas to electrical energy comprising a Brayton cycle and a Carnot cycle for power generation. In the combined cycle operation, the gaseous fuel is combined with an oxygen-bearing gas, combusted, and fed to one or more combustion turbines to generate electrical or mechanical energy. The hot exhaust gases from the combustion turbine or turbines are fed to one or more heat recovery steam generators (HRSG) in which a fraction of the thermal energy in the hot exhaust gases is recovered as steam. The steam from the one or more HRSG's along with any steam generated in other sections of the process (i.e., by recovery of exothermic heat of chemical reactions) is fed to one or more steam turboexpanders to generate electrical or mechanical energy, before rejecting any remaining low level heat in the turbine exhaust to a condensation medium. Numerous variations on the basic combined cycle operation are known in the art. Examples are the HAT (humid air turbine) cycle and the Tophat cycle. All are suitable for use without limitation in the power producing zone of the instant invention. For example, in another embodiment of the invention, the power producing zone may comprise an integrated gasification combined cycle (abbreviated herein as “IGCC”) power plant.

In another aspect of the invention, the blended and unblended syngas streams can be produced in volumes that vary in response to peak and off-peak power demands on a power producing zone. For example, during periods of off-peak power demand, one or more of the combustion turbines which produce electrical power can be shut down. As the combustion turbine is shut down, the portion of raw syngas from step (a) that was consumed by the combustion turbines is instead sent to the water-gas shift reaction zone to produce an increased volume of shifted syngas stream (i) and blended syngas stream (iii) and less of unshifted syngas stream (ii) and unblended syngas stream (iv). A portion of the unshifted syngas stream (ii) is blended with the shifted syngas stream (ii) to produce at least one blended syngas stream (iii) having a hydrogen:carbon monoxide molar ratio that is suitable for the chemical producing zone. For example, a hydrogen:carbon monoxide molar ratio of about 2:1 is needed for methanol production. The blended syngas stream (iii) is then directed to a chemical producing zone. During a period of peak power demand, however, this procedure is reversed and the volume of raw syngas directed to the common water-gas shift reaction zone is reduced and a larger volume of unblended syngas (iv) is produced and sent to the combustion turbine. In this fashion, the throughput of the syngas is kept at a substantially base-loaded value, fully utilizing the expensive syngas-generating equipment, while allowing for the dispatch of a cyclical and variable power loading factor, and maximizing chemical production with syngas not required for power generation. Such a novel combination provides a power generating operation of unusual flexibility, offers substantial economic advantages, and is particularly responsive to present power variation requirements faced by electric power producers. This is in direct contrast to conventional IGCC power plant designs, wherein the power generating facility is operated in base-loaded mode with uneconomical load-following capability. For example, in one embodiment of the invention, a power plant may be operated at 100% of its maximum power producing capacity at peak power demands during the day and fueled entirely by syngas.

“Peak power demand”, as used herein within the context of the present invention, means the maximum power demand on the power producing zone within a given 24 hour period of time. The phrase “period of peak power demand”, as used herein, means one or more intervals of time within the above 24 hour period in which the power demand on the power producing zone is at least 90% of the maximum power demand. “Period of off-peak power demand”, as used herein, means one or more intervals of time within a given 24 hour period in which the power demand on the power producing zone is less than 90% of the peak power demand as defined above.

The chemical producing zone may be used to produce any chemical that is efficiently obtained from a syngas feedstock such as, for example, methanol, alkyl formates, oxo aldehydes, ammonia, dimethyl ether, hydrogen, Fischer-Tropsch products, methane, or a combination of one or more of these chemicals. For example, in one embodiment of the invention, the chemical producing zone is a methanol-producing zone.

The methanol-producing zone can comprise any type of methanol synthesis plant that are well known to persons skilled in the art and many of which are widely practiced on a commercial basis. Most commercial methanol synthesis plants operate in the gas phase at a pressure range of about 25 to about 140 bara using various copper based catalyst systems depending on the technology used. A number of different state-of-the-art technologies are known for synthesizing methanol such as, for example, the ICI (Imperial Chemical Industries) process, the Lurgi process, the Haldor-Topsoe process, and the Mitsubishi process. Liquid phase processes are also well known in the art. Thus, the methanol process according to the present invention may comprise a fixed bed or liquid slurry phase methanol reactor.

The syngas stream is typically supplied to a methanol reactor at the pressure of about 25 to about 140 bara, depending upon the process employed. The syngas then reacts over a catalyst to form methanol. The reaction is exothermic; therefore, heat removal is ordinarily required. The raw or impure methanol is then condensed and may be purified to remove impurities such as higher alcohols including ethanol, propanol, and the like or, burned without purification as fuel. The uncondensed vapor phase comprising unreacted syngas feedstock typically is recycled to the methanol process feed.

The changeover between power production and chemical production is another consideration of the instant invention. For example, when methanol is produced by a gas phase reaction and during periods of no methanol production, flow to the methanol reactor can be greatly reduced or stopped. The reactor can be valved off to contain the gaseous components within the reactor wherein the reactive syngas components will rapidly reach the equilibrium limit of methanol production. The reactor can be kept in this idle state indefinitely. It is desirable, however, to maintain the reactor temperature such that methanol production will start immediately open reintroduction of syngas flow, for example above about 200° C. Surprisingly it has been found that the thermal mass of the catalyst and reactor itself will maintain the temperature above the desired range for several hours, typically four to ten hours, without further heat addition. It may be necessary, however, to provide additional heat input into the idled reactor. The additional heat may be provided by circulation of hot inert gases (for example nitrogen) through the reactor or by contact of a heat transfer medium (for example hot water or steam) to the heat transfer surfaces of the reactor (for example tube walls of a fixed bed tubular reactor) depending on the reactor format used therein.

For liquid phase slurry reactors, it is advantageous to keep the catalyst suspended in the liquid when the methanol reactor is in idle mode, i.e., during periods of peak power demand. An inert gas, for example nitrogen, is fed to the reactor in place of the reactive syngas at a velocity and volume such to prevent settling of the catalyst. Methods for calculating the required flow rate to ensure suspension of the catalyst are well-known in the art. When methanol production is to resume, syngas flow is commenced as the nitrogen flow is reduced. Purge from the reactor, which can be initially high, is decreased to normal levels as the amount of nitrogen drops off.

The thermal mass of a slurry fluid, reactor vessel, and/or catalyst will maintain the temperature above the desired range for several hours, typically four to ten hours, without further heat addition. It may be necessary, however, to provide additional heat input into the idled reactor. The additional heat may be provided by circulation of hot inert gases (for example nitrogen) through the reactor or by contact of a heat transfer medium (for example hot water or steam) to the heat transfer surfaces of the reactor. For example, a portion of at least one of the synthesis gas streams can be passed to the methanol-producing zone during the period of peak power demand to maintain the methanol-producing zone at an elevated temperature through the production of small amounts of methanol. All of the methanol product then can be passed from the methanol-producing zone to the power-producing zone as additional fuel during the period of peak power demand.

Each of the syngas streams (i) and (ii) from step (b) or each of the syngas streams (iii) and (iv) from step (c) can be passed through one or more separate gas cooling zones in which the temperature of syngas is reduced. Gas cooling and recovery of heat energy from the syngas may be accomplished by any means known in the art. For example, the gas cooling zones may comprise at least one of the following types of heat exchangers selected from steam generating heat exchangers (i.e., boilers), wherein heat is transferred from the syngas to boil water; gas-gas interchangers; boiler feed water exchangers; forced air exchangers; cooling water exchangers; direct contact water exchangers; or combinations of one or more of these heat exchangers. The use of multiple steam generating heat exchangers, producing successively lower pressure steam levels is contemplated to be within the scope of the instant invention. Steam and condensate generated within gas cooling zones and may embody one or more steam products of different pressures. The gas cooling zones optionally may comprise other absorption, adsorption, or condensation steps for removal of trace impurities, e.g., such as ammonia, hydrogen chloride, hydrogen cyanide, and trace metals such as mercury, arsenic, and the like.

Our novel process may further comprise passing each of the syngas streams (i) and (ii) from step (b) or each of the syngas streams (iii) and (iv) from step (c) through separate acid gas removal zones in which acidic gases such as, for example, hydrogen sulfide or carbon dioxide, are removed or their concentrations reduced. For example, it is often desirable to remove sulfur-containing compounds present in the syngas in an acid gas removal zone to prevent poisoning of any catalysts when the gas is used for chemical synthesis or to reduce sulfur emissions to the environment when the gas is used for power production. According to the invention, therefore, acid gas removal zones may comprise a sulfur removal zone which may employ any of a number of methods known in the art for removal of sulfur-containing compounds from gaseous streams. The sulfurous compounds may be recovered from the syngas feed to the sulfur removal zone by chemical absorption methods, exemplified by using caustic soda, potassium carbonate or other inorganic bases, or alkanol amines. Examples of suitable alkanolamines for the present invention include primary, secondary, and tertiary amino alcohols containing a total of up to 10 carbon atoms and having a normal boiling point of less than about 250° C. Specific examples include primary amino alcohols such as monoethanolamine (MEA), 2-amino-2-methyl-1-propanol (AMP), 1-aminobutan-2-ol, 2-amino-butan-1-ol, 3-amino-3-methyl-2-pentanol, 2,3-dimethyl-3-amino-1-butanol, 2-amino-2-ethyl-1-butanol, 2-amino-2-methyl-3-pentanol, 2-amino-2-methyl-1-butanol, 2-amino-2-methyl-1-pentanol, 3-amino-3-methyl-1-butanol, 3-amino-3-methyl-2-butanol, 2-amino-2,3-dimethyl-1-butanol, secondary amino alcohols such as diethanolamine (DEA), 2-(ethylamino)-ethanol (EAE), 2-(methylamino)-ethanol (MAE), 2-(propylamino)-ethanol, 2-(isopropylamino)-ethanol, 2-(butylamino)-ethanol, 1-(ethylamino)-ethanol, 1-(methylamino)-ethanol, 1-(propylamino)-ethanol, 1-(isopropylamino)-ethanol, and 1-(butylamino)-ethanol, and tertiary amino alcohols such as triethanolamine (TEA), and methyl-diethanol-amine (MDEA).

Alternatively, sulfur in the syngas feed to the acid gas removal zone may be removed by physical absorption methods. Examples of suitable physical absorbent solvents are methanol and other alkanols, propylene carbonate and other alkyl carbonates, dimethyl ethers of polyethylene glycol of two to twelve glycol units and mixtures thereof (commonly known under the trade name of Selexol™ solvents), n-methyl-pyrrolidone, and sulfolane. Physical and chemical absorption methods may be used in concert as exemplified by the Sulfinol™ process using sulfolane and an alkanolamine as the absorbent, or the Amisol™ process using a mixture of an amine and methanol as the absorbent.

The sulfur-containing compounds may be recovered from the gaseous feed to the sulfur removal zone by solid sorption methods using fixed, fluidized, or moving beds of solids exemplified by zinc titanate, zinc ferrite, tin oxide, zinc oxide, iron oxide, copper oxide, cerium oxide, or mixtures thereof. If necessary for chemical synthesis needs, the chemical or physical absorption processes or solid sorption processes may be followed by an additional method for final sulfur removal. Examples of final sulfur removal processes are adsorption on zinc oxide, copper oxide, iron oxide, manganese oxide, and cobalt oxide.

Typically at least 90 mole percent, more typically at least 95 mole percent, and even more typically, at least 99 mole percent of the total sulfur-containing compounds present in syngas streams (i) and (ii) or (iii) and (iv) are removed in the sulfur removal zone. Typically, syngas used for chemical production requires more stringent sulfur removal, i.e., at least 99.5% removal, to prevent deactivation of chemical synthesis catalysts, more typically the effluent gas from the sulfur removal zone contains less than 5 ppm by volume sulfur.

In addition to sulfur, a portion of the carbon dioxide present may be removed in the acid gas removal zone before passing shifted and blended syngas streams (i) or (iii) to a chemical production zone. Removal or reduction of carbon dioxide may comprise any of a number of methods known in the art. Carbon dioxide in the gaseous feed may be removed by chemical absorption methods, exemplified by using caustic soda, potassium carbonate or other inorganic bases, or alkanol amines. Examples of suitable alkanolamines for the present invention include primary, secondary, and tertiary amino alcohols containing a total of up to 10 carbon atoms and having a normal boiling point of less than about 250° C. Specific examples include primary amino alcohols such as monoethanolamine (MEA), 2-amino-2-methyl-1-propanol (AMP), 1-aminobutan-2-ol, 2-amino-butan-1-ol, 3-amino-3-methyl-2-pentanol, 2,3-dimethyl-3-amino-1-butanol, 2-amino-2-ethyl-1-butanol, 2-amino-2-methyl-3-pentanol, 2-amino-2-methyl-1-butanol, 2-amino-2-methyl-1-pentanol, 3-amino-3-methyl-1-butanol, 3-amino-3-methyl-2-butanol, 2-amino-2,3-dimethyl-1-butanol, and secondary amino alcohols such as diethanolamine (DEA), 2-(ethylamino)-ethanol (EAE), 2-(methylamino)-ethanol (MAE), 2-(propylamino)-ethanol, 2-(isopropylamino)-ethanol, 2-(butylamino)-ethanol, 1-(ethylamino)-ethanol, 1-(methylamino)-ethanol, 1-(propylamino)-ethanol, 1-(isopropylamino)-ethanol, and 1-(butylamino)-ethanol, and tertiary amino alcohols such as triethanolamine (TEA), and methyl-diethanol-amine (MDEA).

Alternatively, carbon dioxide in the gaseous feed may be removed by physical absorption methods. Examples of suitable physical absorbent solvents are methanol and other alkanols, propylene carbonate and other alkyl carbonates, dimethyl ethers of polyethylene glycol of two to twelve glycol units and mixtures thereof (commonly known under the trade name of Selexol™ solvents), n-methyl-pyrrolidone, and sulfolane. Physical and chemical absorption methods may be used in concert as exemplified by the Sulfinol™ process using sulfolane and an alkanolamine as the absorbent, or the Amisol™ process using a mixture of an amine and methanol as the absorbent. If necessary for chemical synthesis needs, the chemical or physical absorption processes may be followed by an additional method for final carbon dioxide removal. Examples of final carbon dioxide removal processes are pressure or temperature-swing adsorption processes.

When required for a particular chemical synthesis process, typically at least 60%, more typically, at least 80% of the carbon dioxide in the feed gas may be removed in the acid gas removal zone. For example, the process of the invention may further comprise removing the carbon dioxide from shifted or blended synthesis gas streams (i) or (iii) to give a carbon dioxide concentration of about 0.5 to about 10 mole %, based on the total moles of gas in the synthesis gas stream, before passing the syngas to the methanol-producing zone. In another example, the carbon dioxide may be removed from at least one of the syngas streams (i) or (iii) to a concentration of about 2 to about 5 mole %. Many of the sulfur and carbon dioxide removal technologies are capable of removing both sulfur and carbon dioxide. Thus, the sulfur and carbon dioxide removal step may be integrated together to simultaneously remove sulfur and carbon dioxide either selectively, (i.e. in substantially separate product streams) or non-selectively, (i.e., as one combined product stream) by means well known in the art.

The acid gas removal zone may be preceded by a gas cooling zone, as described hereinabove, to reduce the temperature of the crude syngas as required by the particular acid gas removal technology utilized therein. Heat energy from the syngas may be recovered through steam generation in the cooling train by means known in the art. The gas cooling zone may optionally comprise other absorption, adsorption, or condensation steps for removal or reaction of trace impurities, e.g., such as ammonia, hydrogen chloride, hydrogen cyanide, trace metals such as mercury, arsenic, and the like. The gas cooling zone, optionally, may comprise a reaction step for converting carbonyl sulfide to hydrogen sulfide and carbon dioxide via reaction with water.

Another embodiment of our invention is a process for producing variable syngas compositions, comprising:
(a) reacting an oxidant stream with coal or petroleum coke in a gasification zone comprising at least 2 gasifiers to produce at least 2 raw syngas streams comprising carbon monoxide, hydrogen, carbon dioxide, and sulfur-containing compounds,
(b) passing a portion of at least one of the raw syngas streams from step (a) to a common water-gas shift reaction zone to produce at least one shifted syngas stream (i) having a molar ratio of hydrogen to carbon monoxide of about 1:1 to about 20:1, and at least one unshifted syngas stream (ii), comprising a remaining portion of the raw syngas streams;
(c) generating steam in the water-gas shift reaction zone by recovery of heat from the shifted syngas stream (i);
(d) combining a portion of the steam from step (c) with the portion of one or more raw syngas streams before passing to the water-gas shift reaction zone;
(e) blending the shifted syngas stream (i) with a portion of the unshifted syngas stream (ii) to produce at least one blended syngas stream (iii) and at least one unblended syngas stream (iv) comprising a remaining portion of unshifted syngas stream (ii); and
(f) passing blended gas stream (iii) to a methanol or dimethyl ether producing zone and unblended gas stream (iv) to a power producing zone.
   It is understood that the above process comprises the various embodiments of the gasifier, syngas streams, steam generation, oxidant stream, carbonaceous materials, power-producing zone, acid-gas removal zones, and gas cooling zones as described hereinabove. For example, the process may further comprise passing each of the syngas streams (i) and (ii) from step (b) or each of the syngas streams (iii) and (iv) from step (e) through separate gas cooling zones. Each of syngas streams (i) and (ii) or (iii) and (iv) also may be passed through separate acid gas removal zones, comprising a sulfur removal zone, a carbon dioxide removal zone, or a combination thereof. The process may further comprise removing at least 95 mole percent of the total sulfur-containing compounds present in the syngas streams (i) and (ii) or (iii) and (iv) in a sulfur removal zone and/or a portion of the carbon dioxide from syngas stream (iii) in a carbon dioxide removal zone.

The blended syngas stream can be passed to chemical producing zone which can comprise either a methanol or dimethyl ether process, and the unblended gas can be passed to a power producing zone to produce electrical power as described previously. For example, the blended and unblended syngas streams (iii) and (iv) may be produced in volumes that vary in response to peak and off-peak power demands on the power producing zone. In this embodiment, for example, the volume of blended syngas stream (iii) is increased during periods of off-peak power demand and used to produce methanol or dimethyl ether while the volume of unblended syngas stream (iv) is decreased. Conversely, during periods of peak power demand, the volume of unblended syngas stream (iv) is increased and used as fuel for a power-producing zone, while the volume of blended syngas stream (iii) is decreased.

Our invention also provides a process for producing variable volumes of power and methanol, comprising:
(a) reacting an oxidant stream with coal or petroleum coke in a gasification zone comprising at least 2 gasifiers to produce at least 2 raw syngas streams comprising carbon monoxide, hydrogen, carbon dioxide, and sulfur-containing compounds,
(b) passing a portion of at least one of the raw syngas streams from step (a) to a common water-gas shift reaction zone to produce at least one shifted syngas stream (i) having an enriched hydrogen content, and at least one unshifted syngas stream (ii), comprising a remaining portion of the raw syngas streams;
(c) blending the shifted syngas stream (i) with up to 100 volume percent of the unshifted syngas stream (ii) to produce at least one blended syngas stream (iii) and a remaining portion of the unshifted syngas stream (ii);
(d) producing methanol by passing the blended gas stream (iii) from step (c) to a methanol producing zone; and
(e) passing the remaining portion of unshifted syngas stream (ii) to a power producing zone to produce electrical power;
wherein the blended syngas stream is produced in a quantity that varies in response to periods of peak and off-peak power demands on the power producing zone.
   As noted above, the process includes the various embodiments of the gasifier, syngas streams, steam generation, oxidant stream, carbonaceous materials, power-producing zone, acid gas-removal zones, and cooling zones as described previously. For example, the gasifiers can be used to oxidize carbonaceous material such as coal or petroleum coke to syngas and can be sized to supply at least 90% of the maximum capacity fuel requirements of the power-producing zone. The purity of oxidant stream typically is at least 85 volume % oxygen, and may comprise at least 95 volume % oxygen or, in another example at least 98 volume % oxygen. The methanol producing zone is as described previously and may comprise, for example, a fixed bed or liquid slurry phase methanol reactor.

As described previously, steam may be generated the water-gas shift reaction zone by recovering heat from the shifted syngas stream (i) as it exits the water gas-shift reaction zone and before blending with the unshifted syngas stream (ii) in step (c). The steam generated in the water-gas shift reaction zone may be directed to a common steam header and used as general utility steam or can be used to add sufficient water to the raw syngas entering the water-gas shift reaction zone by combining a portion of the steam with a the portion of one or more raw syngas streams in step (b) from the gasification zone to produce at least one wet syngas stream and passing that wet syngas stream to the water-gas shift reaction zone. Typically, the molar ratio of water to carbon monoxide in the wet syngas stream is about 1.5:1 to about 3:1. Additional examples of water:carbon monoxide molar ratios that may be produced are 2:1 and 2.5:1.

Each of the syngas streams present in steps (a), (b), or (c) can be passed through separate gas cooling zones and/or separate acid gas removal zones. The acid gas removal zones can comprise a sulfur removal zone, a carbon dioxide removal zone, or a combination thereof. For example, at least 95 mole percent of the total sulfur-containing compounds present in the syngas streams present in steps (a), (b), or (c) are in a sulfur removal zone. In another embodiment, the process may further comprise removing a portion the carbon dioxide from syngas streams (i) or (iii) to give a carbon dioxide concentration of about 0.5 to about 10 mole %, based on the total moles of gas in syngas streams (i) or (iii), before passing to the methanol-producing zone of step (d).

The blended syngas stream (iii) may be produced in a quantities that vary in response to periods of peak and off-peak power demands on the power producing zone by adjusting the volume of raw syngas that is passed to the water-gas shift reaction zone and the volume of unshifted syngas (ii) that is blended with the shifted syngas stream (i). Up to 100 volume percent of the unshifted syngas stream (ii) may be blended with the shifted syngas stream (ii). For example, 100 volume percent of the unshifted syngas stream (ii) may be blended with the shifted syngas stream (i) during a period of off-peak power demand. In this embodiment, the entire volume of unshifted syngas stream (ii) can blended with shifted syngas stream (i) to make the blended syngas stream (iii) instead of passed to the power-producing zone. The blended syngas stream is passed to a methanol producing zone and used to produce methanol.

In another example, the power producing zone may comprise at least one combustion turbine which may be shut down during a period of off-peak power demand. In response to the lower power demand on the power producing zone, the volume of raw syngas directed to the water gas shift reaction zone can be increased and 100 volume % of the unshifted syngas stream can be blended with the shifted syngas stream. The blended syngas stream is then passed to the methanol producing zone. There may be more than one period of off-peak power demand within a 24 hour period. Thus, a combustion turbine may be shut down more than one time within a given 24 hour period. By shutting down at least one combustion turbine during these periods of off-peak power demand instead of operating the turbine in an inefficient or uneconomical regime, the gasifier can be operated efficiently as a constant rate and the maximum thermodynamic and economic value of the syngas realized.

For example, a power producing zone comprising two combustion turbines, might operate at 90% or greater of full capacity. As demand for power drops, it can be advantageous for economic reasons (i.e., low price of power) or because of thermodynamic inefficiency to shut one or more combustion turbines. Therefore, according to the process of the invention, rather than continue to operate one of the turbines in an inefficient and/or uneconomical manner and, the turbine is shut down and synthesis gas feed stream passed instead to a chemical producing zone to produce chemicals. Thus, instead of using the syngas stream to produce electrical power with a turbine operating at an inefficient capacity factor, the syngas is used to produce chemicals which may be, for example, sold on the market or used to supplement the fuel requirements of the combustion turbines. In addition to methanol, it is within the scope of the present invention to produce any chemical that is efficiently obtained from a syngas feedstock such as, for example, methanol, alkyl formates, oxo aldehydes, methane, ammonia, dimethyl ether, hydrogen, Fischer-Tropsch products, or a combination of one or more of these chemicals.

In one embodiment of the invention, for example, ammonia and/or hydrogen can be produced in the chemical producing zone. In this example, the water gas shift reaction zone would be operated to maximize hydrogen and carbon dioxide production. Typical conversions of carbon monoxide to hydrogen and carbon dioxide are greater than 95%. The carbon dioxide removal zone may comprise conventional absorption or adsorption technologies described above, followed by final purification step. For example pressure swing adsorption, wherein the oxygenate content of the hydrogen is reduced to less than 2 ppm by volume. The hydrogen can be sold or used to produce ammonia in the chemical producing zone by the Haber-Bosch process by means known in the art as exemplified by LeBlance et al in “Ammonia”, Kirk-Othmer Encyclopedia of Chemical Technology, Volume 2, 3^{rd} Edition, 1978, pp. 494-500.

In another embodiment of the invention, Fischer-Tropsch products such as, for example, hydrocarbons and alcohols, can be produced in the chemical producing zone via a Fischer-Tropsch reaction as exemplified in U.S. Pat. Nos. 5,621,155 and 6,682,711. Typically, the Fischer-Tropsch reaction may be effected in a fixed bed, in a slurry bed, or in a fluidized bed reactor. The Fischer-Tropsch reaction conditions may include using a reaction temperature of between 190° C. and 340° C., with the actual reaction temperature being largely determined by the reactor configuration. For example, when a fluidized bed reactor is used, the reaction temperature is preferably between 300° C. and 340° C.; when a fixed bed reactor is used, the reaction temperature is preferably between 200° C. and 250° C.; and when a slurry bed reactor is used, the reaction temperature is preferably between 190° C. and 270° C.

An inlet syngas pressure to the Fischer-Tropsch reactor of between 1 and 50 bar, preferably between 15 and 50 bar, may be used. The syngas may have a H₂:CO molar ratio, in the fresh feed, of 1.5:1 to 2.5:1, preferably 1.8:1 to 2.2:1. The synthesis gas typically includes 0.1 wppm of sulfur or less. A gas recycle may optionally be employed to the reaction stage, and the ratio of the gas recycle rate to the fresh synthesis gas feed rate, on a molar basis, may then be between 1:1 and 3:1, preferably between 1.5:1 and 2.5:1. A space velocity, in m³ (kg catalyst)⁻¹ hr⁻¹, of from 1 to 20, preferably from 8 to 12, may be used in the reaction stage.

In principle, an iron-based, a cobalt-based or an iron/cobalt-based Fischer-Tropsch catalyst can be used in the Fischer-Tropsch reaction stage, although Fischer-Tropsch catalysts operated with high chain growth probabilities (i.e., alpha values of 0.8 or greater, preferably 0.9 or greater, more preferably, 0.925 or greater) are typical. Reaction conditions are preferably chosen to minimize methane and ethane formation. This tends to provide product streams which mostly include wax and heavy products, i.e., largely paraffinic C₂₀+linear hydrocarbons.

The iron-based Fischer-Tropsch catalyst may include iron and/or iron oxides which have been precipitated or fused. However, iron and/or iron oxides which have been sintered, cemented, or impregnated onto a suitable support can also be used. The iron should be reduced to metallic Fe before the Fischer-Tropsch synthesis. The iron-based catalyst may contain various levels of promoters, the role of which may be to alter one or more of the activity, the stability, and the selectivity of the final catalyst. Typical promoters are those influencing the surface area of the reduced iron (“structural promoters”), and these include oxides or metals of Mn, Ti, Mg, Cr, Ca, Si, Al, or Cu or combinations thereof.

The products from Fischer-Tropsch reactions often include a gaseous reaction product and a liquid reaction product. For example, the gaseous reaction product typically includes hydrocarbons boiling below about 343° C. (e.g., tail gases through middle distillates). The liquid reaction product (the condensate fraction) includes hydrocarbons boiling above about 343° C. (e.g., vacuum gas oil through heavy paraffins) and alcohols of varying chain lengths.

The chemical producing zone also may be used to produce oxo aldehydes using hydroformylation processes that are well known in the art. The hydroformylation reaction is typically carried out by contacting an olefin such as, for example, ethylene or propylene, with carbon monoxide and hydrogen in the presence of a transition metal catalyst to produce linear and branched aldehydes. Examples of aldehydes that can be produced by hydroformylation include acetaldehyde, butyraldehyde, and isobutyraldehyde.

In another example, alkyl formates such as, for example, methyl formate are produced in the chemical producing zone. There are currently several known processes for the synthesis of alkyl formates such as methyl formate from a syngas and alkyl alcohol feedstock. In addition to U.S. Pat. No. 3,716,619, they include U.S. Pat. No. 3,816,513, wherein carbon monoxide and methanol are reacted in either the liquid or gaseous phase to form methyl formate at elevated pressures and temperatures in the presence of an alkaline catalyst and sufficient hydrogen to permit carbon monoxide to be converted to methanol, and U.S. Pat. No. 4,216,339, in which carbon monoxide is reacted at elevated temperatures and pressures with a current of liquid reaction mixture containing methanol and either alkali metal or alkaline earth metal methoxide catalysts to produce methyl formate. In the broadest embodiment of this invention, however, any effective commercially viable process for the formation of an alkyl formate from a feedstock comprising a corresponding alkyl alcohol and a prepared syngas sufficiently rich in carbon monoxide is within the scope of the invention. The precise catalyst or catalysts chosen, as well as concentration, contact time, and the like, can vary widely, as is known to those skilled in the art. It is preferred to use the catalysts disclosed in U.S. Pat. No. 4,216,339, but a wide variety of other catalysts known to those in the art can also be used.

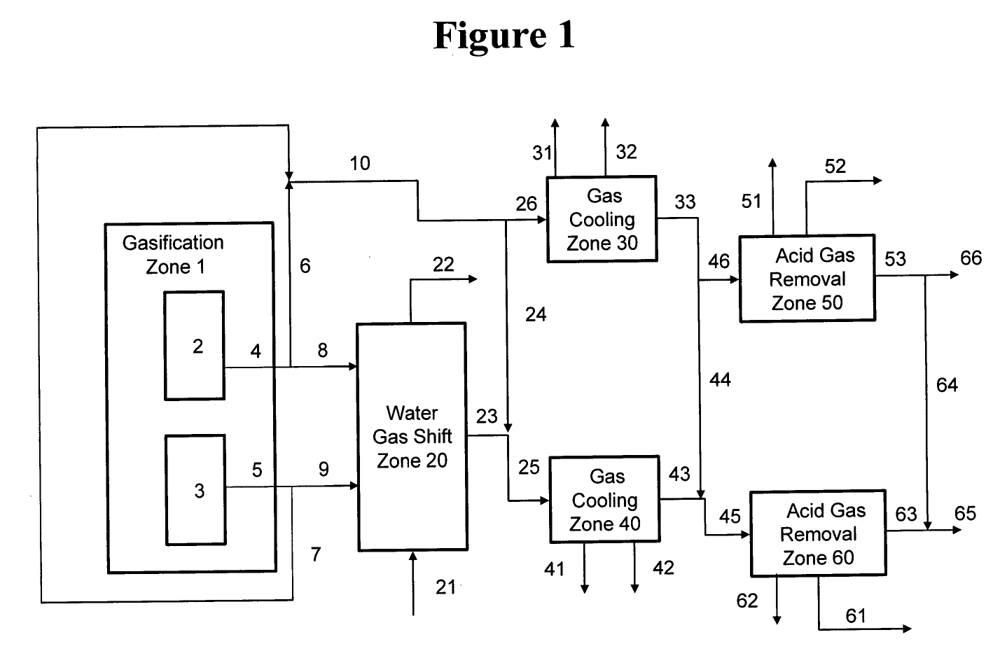

A better understanding of one embodiment of the invention is provided with particular reference to the process flow diagram depicted in FIG. 1. In the embodiment set forth in FIG. 1, hydrocarbonaceous or carbonaceous materials are gasified in gasification zone 1 comprising two or more gasifiers of any type known in the art (shown as gasifiers 2 and 3 in FIG. 1) to produce crude syngas streams 4 and 5. The flow of syngas streams 4 and 5 is divided between conduits 6, 7, 8, and 9 by flow control methods known in the art, wherein the ratio of flow of streams 6 and 8 to 7 and 9 is dependent on the desired compositions and volumes of product streams 65 and 66. The fraction of gas directed to conduits 8 and 9 may vary from 0-100% of the flows of conduits 4 and 5 respectively. Streams 6 and 7 are combined in conduit 10 to produce an unshifted gas stream.

The fraction of the gas directed via conduits 8 and 9 is passed to a common water-gas shift reaction zone 20 wherein the gas undergoes the equilibrium-limited water-gas shift reaction in which carbon monoxide is reacted with water to produce hydrogen and carbon dioxide. The steam generated by the heat of the exothermic shift reaction exits the water-gas shift zone via conduit 22. Depending on the water content of the raw syngas from gasification zone 1, it may be necessary to add water or steam directly to streams 8 and 9 via conduit 21 to provide sufficient water to carry out properly the water gas shift reaction. Typically the molar ratio of CO to vaporous water in the combined feed to water gas shift zone 20 is greater than or equal to 1.5 to 1, more preferably greater than or equal to 2 to 1. If desired, all or part of the steam added via conduit 21 to shift zone 20 may be supplied from that generated within the shift zone itself, i.e., via conduit 22, provided the pressure of conduit 22 is greater than or equal to the pressure of conduits 8 and 9.

The shifted gas is conveyed via conduits 23 and 25 to gas cooling zone 40 wherein the temperature of the gas is reduced and the gas is prepared for further purification. Heat energy from the syngas may be recovered through any means known in the art. Gas cooling zone 40 may comprise any or all of the following types of heat exchangers: steam generating heat exchangers (i.e., boilers) wherein heat is transferred from the syngas to boil water, gas-gas interchangers, boiler feed water exchangers, forced air exchangers, cooling water exchangers, and direct contact water exchangers. The use of multiple steam generating heat exchangers, producing successively lower pressure steam levels is contemplated to be within the scope of the instant invention. Steam and condensate generated within gas cooling zones 30 and 40 exit via conduits 31, 32 and 41, 42 respectively. It is understood that conduit 41 may embody one or more steam products of different pressures. Gas cooling zone 40 may optionally comprise other absorption, adsorption, or condensation steps for removal of trace impurities, such as ammonia, hydrogen chloride, hydrogen cyanide, and trace metals such as mercury, arsenic, and the like. In addition, gas cooling zone 40 may optionally comprise a reaction step for converting carbonyl sulfide to hydrogen sulfide and carbon dioxide via reaction with water.

The unshifted gas is conveyed via conduits 10 and 26 to gas cooling zone 30 wherein the temperature of said gas is reduced and said gas is prepared for further purification. Heat energy from the syngas may be recovered through any means known in the art. Gas cooling zone 30 may comprise any or all of the following types of heat exchangers: steam generating heat exchangers (i.e., boilers) wherein heat is transferred from the syngas to boil water, gas-gas interchangers, boiler feed water exchangers, forced air exchangers, cooling water exchangers, and direct contact water exchangers. The use of multiple steam generating heat exchangers, producing successively lower pressure steam levels is contemplated to be within the scope of the instant invention. Steam and condensate generated within gas cooling zone 30 exits via conduits 31 and 32 respectively. It is understood that conduit 31 may embody one or more steam products of different pressures. Gas cooling zone 30 may optionally comprise other absorption, adsorption, or condensation steps for removal of trace impurities, e.g., such as ammonia, hydrogen chloride, hydrogen cyanide, and trace metals such as mercury, arsenic, and the like. Gas cooling zone 30 may optionally comprise a reaction step for converting carbonyl sulfide to hydrogen sulfide and carbon dioxide via reaction with water. The cooled, unshifted syngas can be passed to acid gas removal zone 50 to remove all or a portion of the sulfur and/or carbon dioxide, or all or a portion may be passed to the stream blended with the cooled shifted syngas via conduit 44.

The cooled, shifted gas is conveyed via conduits 43 and 45 to acid gas removal zone 60 wherein all or a portion of the acid gas components of crude syngas are removed, e.g. hydrogen sulfide, carbonyl sulfide, and carbon dioxide. Similarly, cooled, shifted gas is conveyed via conduits 33 and 46 to acid gas removal zone 60 wherein the acid gas components of the crude syngas are removed, e.g. hydrogen sulfide, carbonyl sulfide, and optionally carbon dioxide. Streams 51 and 61 are rich in recovered sulfur-bearing species and, optionally, streams 52 and 62 are rich in carbon dioxide.

Typically different removal levels of sulfur-bearing species and carbon dioxide are required for different syngas applications. For example, environmental regulations on acid gas emissions from power generating plants typically limit sulfur content of the cleaned syngas to less 100 parts per million by volume with carbon dioxide levels currently unregulated, whereas much less than 1 part per million sulfur content and typically less than 5 mole percent carbon dioxide content are required in order to ensure proper operation and lifetime of a methanol synthesis catalyst. Many other chemical synthesis catalysts, e.g., Fischer-Tropsch, ammonia, oxo, and methanation catalysts have similar or more stringent restrictions on acid gas content than methanol catalysts. Therefore, zones 50 and 60 may be designed for different acid gas removal specifications.

Sulfur-bearing species in streams 51 and 61 may be further processed to produce elemental sulfur by any methods known in the art, for example the Claus reaction. Alternatively the sulfur may be oxidized and combined with water to produce sulfuric acid by means well known in the art.

Conduits 24, 44, and 64 are provided for blending of shifted and unshifted syngas streams. Typically the shifted and unshifted syngas streams are blended to produce the blended syngas stream after the acid gas removal zones, i.e., via conduit 64. All or a portion of the sweet syngas can then be used to blend with the shifted syngas stream via conduit 64 or passed to a power producing zone as a fuel for a combustion turbine.

We claim:

1. A process for producing variable syngas compositions, comprising:
(a) reacting an oxidant stream with a carbonaceous material in a gasification zone comprising at least 2 gasifiers to produce at least 2 raw syngas streams comprising carbon monoxide, hydrogen, carbon dioxide, and sulfur-containing compounds,
(b) passing a portion of at least one of said raw syngas streams from step (a) to a common water-gas shift reaction zone to produce at least one shifted syngas stream (i) having an enriched hydrogen content, and at least one unshifted syngas stream (ii), comprising a remaining portion of said raw syngas streams; and
(c) blending said shifted syngas stream (i) with a portion of said unshifted syngas stream (ii) to produce at least one blended syngas stream (iii) and at least one unblended syngas stream (iv) comprising a remaining portion of unshifted syngas stream (ii) wherein said blended syngas stream is produced in a volume and/or composition that varies in response to at least one downstream syngas requirement.

2. The process according to claim 1 further comprising generating steam in said water-gas shift reaction zone.

3. The process according to claim 2 further comprising combining a portion of said steam from said water-gas shift reaction zone with said portion in step (b) of one or more raw syngas streams to produce at least one wet syngas stream and passing said wet syngas stream to said water-gas shift reaction zone.

4. The process according to claim 3 wherein the molar ratio of water to carbon monoxide in said wet syngas stream is about 1.5:1 to about 3:1.

5. The process according to claim 2 wherein said steam is generated by recovery of heat from said shifted syngas stream (i) before step (c).

6. The process according to claim 1 wherein said oxidant stream comprises at least 85 volume % oxygen, based on the total volume of said oxidant stream.

7. The process according to claim 6 wherein said oxidant stream comprises at least 95 volume % oxygen.

8. The process according to claim 1 wherein the carbonaceous material is coal or petroleum coke.

9. The process according to claim 1 further comprising passing each of said syngas streams (i) and (ii) from step (b) or each of said syngas streams (iii) and (iv) from step (c) through separate gas cooling zones.

10. The process according to claim 1 further comprising passing each of said syngas streams (i) and (ii) from step (b) or each of said syngas streams (iii) and (iv) from step (c) through separate acid gas removal zones.

11. The process according to claim 10 wherein said acid gas removal zones comprise a sulfur removal zone in which at least 95 mole percent of the total of said sulfur containing compounds present in said syngas streams (i) and (ii) or (iii) and (iv) are removed.

12. The process according to claim 11 wherein said downstream syngas requirement comprises a feedstock need of a least one chemical process, a fuel need of at least one power plant, or a combination thereof.

13. The process according to claim 1 further comprising (d) passing said blended syngas stream (iii) to a chemical producing zone and said unblended syngas stream (iv) to a power producing zone.

14. The process according to claim 13 wherein said chemical producing zone produces methanol, alkyl formates, dimethyl ether, oxo aldehydes, ammonia, methane, hydrogen, Fischer-Tropsch products, or a combination thereof.

15. The process according to claim 14 wherein said chemical producing zone is a methanol producing zone.

16. The process according to claim 15 further comprising removing a portion of said carbon dioxide from said syngas streams (i) or (iii) to give a carbon dioxide concentration of about 0.5 to about 10 mole %, based on the total moles of gas in said syngas streams (i) or (iii), before passing to said methanol-producing zone of step (d).

17. The process according to claim 13 wherein said power producing zone comprises a combined cycle system.

18. The process according to claim 13 wherein said volume and/or composition of said blended syngas stream varies in response to peak and off-peak power demands.

19. A process for producing variable syngas compositions, comprising:
(a) reacting an oxidant stream with coal or petroleum coke in a gasification zone comprising at least 2 gasifiers to produce at least 2 raw syngas streams comprising carbon monoxide, hydrogen, carbon dioxide, and sulfur-containing compounds,
(b) passing a portion of at least one of said raw syngas streams from step (a) to a common water-gas shift reaction zone to produce at least one shifted syngas stream (i) having a molar ratio of hydrogen to carbon monoxide of about 1:1 to about 20:1, and at least one unshifted syngas stream (ii), comprising a remaining portion of said raw syngas streams;
(c) generating steam in said water-gas shift reaction zone by recovery of heat from said shifted syngas stream (i);
(d) combining a portion of said steam from step (c) with said portion of one or more raw syngas streams before passing to said water-gas shift reaction zone;
(e) blending said shifted syngas stream (i) with a portion of said unshifted syngas stream (ii) to produce at least one blended syngas stream (iii) and at least one unblended syngas stream (iv) comprising a remaining portion of unshifted syngas stream (ii); and
(f) passing said blended syngas stream (iii) to a methanol or dimethyl ether producing zone and unblended syngas stream (iv) to a power producing zone.

20. The process according to claim 19 further comprising passing each of said syngas streams (i) and (ii) from step (b) or each of said syngas streams (iii) and (iv) from step (e) through separate gas cooling zones.

21. The process according to claim 19 further comprising passing each of said syngas streams (i) and (ii) from step (b) or each of said syngas streams (iii) and (iv) from step (e) through separate acid gas removal zones, comprising a sulfur removal zone, a carbon dioxide removal zone, or a combination thereof.

22. The process according to claim 21 further comprising removing at least 95 mole percent of the total sulfur-containing compounds present in said syngas streams (i) and (ii) or (iii) and (iv) in a sulfur removal zone.

23. The process according to claim 21 further comprising removing a portion of said carbon dioxide from syngas stream (iii) in a carbon dioxide removal zone.

24. The process according to claim 19 wherein said blended syngas stream (iii) is produced in a volume and/or composition that varies in response to peak and off-peak power demands.

25. A process for producing variable amounts of power and methanol, comprising:
(a) reacting an oxidant stream with coal or petroleum coke in a gasification zone comprising at least 2 gasifiers to produce at least 2 raw syngas streams comprising carbon monoxide, hydrogen, carbon dioxide, and sulfur-containing compounds,
(b) passing a portion of at least one of said raw syngas streams from step (a) to a common water-gas shift reaction zone to produce at least one shifted syngas stream (i) having an enriched hydrogen content, and at least one unshifted syngas stream (ii), comprising a remaining portion of said raw syngas streams;
(c) blending said shifted syngas stream (i) with up to 100 volume percent of said unshifted syngas stream (ii) to produce at least one blended syngas stream (iii) and a remaining portion of said unshifted syngas stream (ii);
(d) producing methanol by passing said blended gas stream (iii) from step (c) to a methanol producing zone; and
(e) passing the remaining portion of unshifted syngas stream (ii) to a power producing zone to produce electrical power;
wherein said blended syngas stream is produced in a volume and/or composition that varies in response to periods of peak and off-peak power demands on said power producing zone.

26. The process according to claim 25 further comprising generating steam in said water-gas shift reaction zone by recovery of heat from said shifted syngas stream (i) before step (c).

27. The process according to claim 26 further comprising combining a portion of said steam from said water-gas shift reaction zone with said portion in step (b) of one or more raw syngas streams to produce at least one wet syngas stream and passing said wet syngas stream to said water-gas shift reaction zone.

28. The process according to claim 25 wherein said methanol producing zone comprises a fixed bed methanol reactor.

29. The process according to claim 25 wherein said methanol producing zone comprises a liquid slurry phase methanol reactor.

30. The process according to claim 25 wherein said 2 or more gasifiers are sized to supply at least 90% of the maximum capacity fuel requirements of said power-producing zone.

31. The process according to claim 25 further comprising passing each of said syngas streams present in steps (a), (b), or (c) through separate gas cooling zones.

32. The process according to claim 25 further comprising passing each of said syngas streams present in steps (a), (b), or (c) through separate acid gas removal zones.

33. The process according to claim 32 wherein said acid gas removal zones comprise a sulfur removal zone in which at least 95 mole percent of said sulfur containing compounds present in said syngas streams are removed.

34. The process according to claim 25 further comprising removing a portion of said carbon dioxide from said syngas streams (i) or (iii) to give a carbon dioxide concentration of about 0.5 to about 10 mole %, based on the total moles of gas in said syngas streams (i) or (iii), before passing to said methanol-producing zone of step (d).

35. The process according to claim 25 wherein 100 volume percent of said unshifted syngas stream (ii) is blended with said shifted syngas stream (i) during a period of off-peak power demand.
